(19) United States
(12) Patent Application Publication
(10) Pub. No.: US 2016/0143781 A1
(43) Pub. Date: May 26, 2016

(54) VALVE DEVICE FOR USE IN GLAUCOMA SURGERY

(71) Applicant: MICHELE VETRUGNO, BARI (IT)

(72) Inventor: MICHELE VETRUGNO, BARI (IT)

(21) Appl. No.: 14/899,588

(22) PCT Filed: Jun. 19, 2014
(86) PCT No.: PCT/IB2014/062435

§ 371 Date: Dec. 18, 2015

(30) Foreign Application Priority Data
Jun. 25, 2013 (IT) MI2013A001058

(51) Int. Cl. A61F 9/007; A61F 2/14

(52) U.S. Cl. CPC A61F 9/00781; A61F 2/15

(57) ABSTRACT

A valve device for use in glaucoma surgery comprises at least one aqueous humor feeding duct (2) having a first end (2a) designed to fit into the anterior chamber of the eyeball, a balancing chamber (3) communicating with a second end (2b) of the feeding duct (2) and having an inner volume ranging from 2 mm3 to 6 mm3, and designed to be introduced into a surgical groove in the sclera, a retainer member (6) placed within the feeding duct (2) to allow fluid transfer from the first end (2a) toward the second end (2b) of the feeding duct (2) and to inhibit fluid transfer in the opposite direction. A plurality of aqueous humor outflow ducts (5) extend from the balancing chamber (3) to allow outflow of aqueous humor from the balancing chamber (3) to an outside space.

### TECHNICAL FIELD

The present invention relates to a valve device for use in glaucoma surgery and a method for surgical treatment of glaucoma using such device.

### BACKGROUND ART

Glaucoma is the second leading cause of blindness worldwide. 60 million people suffer from such disease and about 32% of them unfortunately incurs blindness. High eye pressure is the first risk factor that has been found, as glaucoma occurrence progressively increases in subjects with increased eye pressures.

Pharmacological and surgical treatments have been developed to reduce eye pressure. Pharmacological treatments include administration of hypotensive eye drops which have the purpose of reducing production of aqueous humor or increasing removal thereof. Nevertheless, pharmacological treatment requires active collaboration by the patient and involves daily administration of eye drops.

Intraocular pressure may be also reduced by laser therapy, i.e. trabeculoplasty, which provides good results but for limited periods of time, after which the disease reappears.

Concerning surgical treatment, standard procedures have the purpose of opening a passage through the eye walls to allow aqueous humor to escape from within the eyeball. The most common procedure among those that may be used for this purpose is trabeculectomy. This surgical procedure is maybe the oldest eye surgery procedure that is still in use. It consists in cutting a small trapdoor flap in the sclera (the outer coat of the eye), allowing communication between the anterior chamber (the interior of the eye) and the exterior of the eye, by forming a pocket in the subconjunctival space.

This surgical technique is still deemed to be the last treatment option, as it involves a number of problems both concerning methods (it is a non-standardized surgical technique) and post-surgery complications. In this respect, the most common late post-surgical complication is reduced filtration caused by normal scleral and conjunctival scarring. This normal process is counteracted using particular substances (mitotic inhibitors), which inhibit proliferation of scar tissue or sometimes by enlarging the communication passage between the anterior chamber and the subconjunctival space. Nevertheless, this would cause over-filtration in the first days, which might initially lead to a dramatic drop of intraocular pressure.

In recent years a number of valve devices and devices of other kind have been introduced into the market, in an attempt to provide standardizable surgical procedures and more predictable results. Some of these act directly upon the sclerocorneal trabecular meshwork (i.e. the region in which aqueous humor is filtered out of the eye) and attempt to bypass it, whereas others generate well-defined filtration volumes, thereby reducing post-surgery complications of trabeculectomy. The drawback of these techniques is their inability to reduce or prevent short-to-medium term scarring of the scleral flap and the conjunctiva, which at most times neutralize the hypotensive effect of the surgical procedure.

In this context, the technical purpose of the present invention is to provide a valve device for use in glaucoma surgery that is free of the above mentioned drawbacks.

Particularly, the object of the present invention is to provide a valve device for use in glaucoma surgery that can afford optimal intraocular pressure recover and minimize tissue scarring problems.

A further object of the present invention is to provide a method for surgical treatment of glaucoma that uses such device,

According to the present invention, these and other objects, as better illustrated hereinafter, are fulfilled by a valve device for use in glaucoma surgery and a method for surgical treatment of glaucoma that uses such device having the features as set forth in one or more of the annexed claims.

### BRIEF DESCRIPTION OF THE DRAWINGS

The accompanying figures show exemplary embodiments of the present disclosure, by way of illustration and without limitation, and:

FIG. 1 shows a perspective view of the valve device for use in glaucoma surgery according to the present invention;

FIG. 2 shows a variant embodiment of the valve device of FIG. 1; and

FIGS. 3 and 4 show sectional views of details of the device of FIG. 1.

### DETAILED DESCRIPTION

As used herein and in the annexed claims, the terms aqueous humor, sclera, trabecular meshwork, limbus, conjunctiva, anterior chamber are intended in their usual anatomical meaning. Particularly, aqueous humor is a saline liquid that fills the space between the cornea and the crystalline; the sclera is an opaque fibrous membrane that forms ⅚ of the outer tunic of the eyeball; the trabecular meshwork is the region between the iris and the cornea, at 360 degrees, through which aqueous humor is drained; the limbus is the junction area between the cornea and the sclera; the conjunctiva is a mucous membrane that covers the posterior surface of the eyelids and the outer surface of the sclera; the anterior chamber is the intraocular space delimited at the front bu the posterior surface of the cornea and at the back by the anterior surface of the iris.

Referring to the accompanying figures, a valve device for use in glaucoma surgery according to the present invention is generally designated by numeral 1. The valve device as shown in the annexed figures has been represented schematically and ratio and/or scale factors are not necessarily applied to the parts of the device.

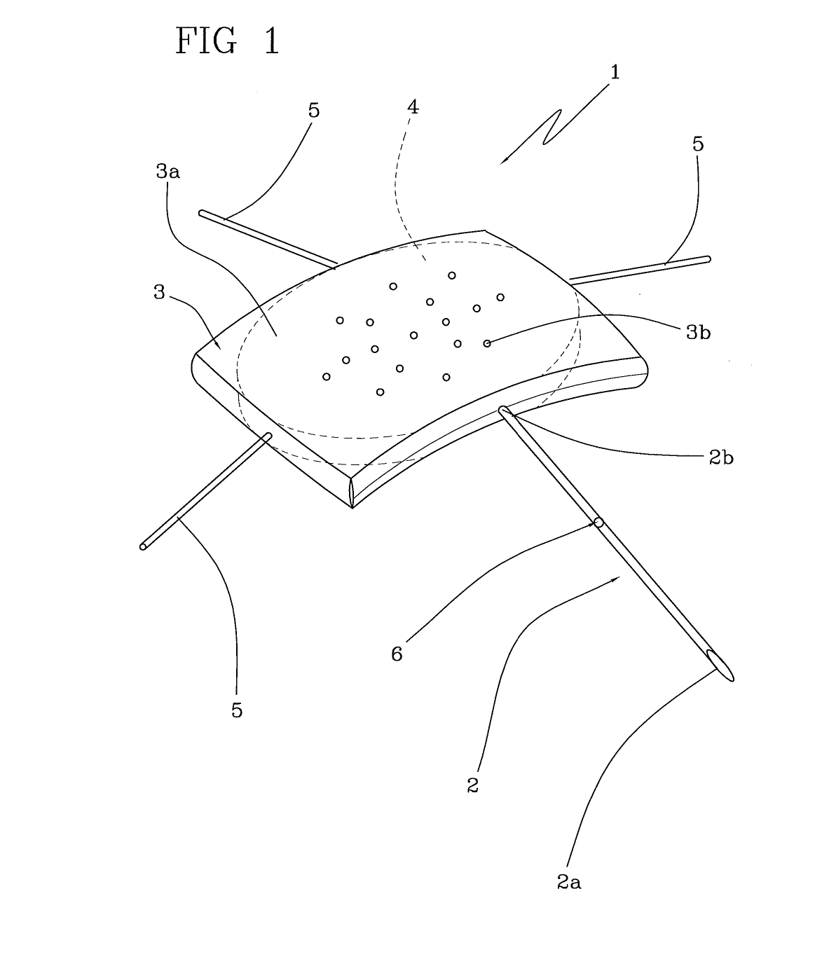

The device 1 comprises at least one aqueous humor feeding duct 2 having a first end 2a designed to fit into the anterior chamber of an eyeball and extend past the limbus. Such first end 2a has a pointed section for insertion thereof into a hole formed in the limbus at the trabecular meshwork such that it may reach the anterior chamber of the eyeball. The feeding duct 2 has a second end 2b which is in fluid communication with an inner volume 4 (outlined by broken lines in FIGS. 1 and 2) of a balancing chamber 3. The diameter of the feeding duct ranges from 20 to 100 micrometers (i.e. μtm), and is preferably about 50 micrometers.

The balancing chamber 3 is designed to fit into a recess (or pocket) of the sclera under the conjunctiva. The inner volume of the balancing chamber 3 preferably ranges from 2 mm³ to 6 mm³, and is preferably about 4 mm³. The outer dimensions of the balancing chamber are preferably about 4 mm×4 mm with a height of about 0.25 mm. The thickness of the walls of the balancing chamber 3 ranges from 10 to 50 micrometers, and is preferably about 20-30 micrometers.

A plurality of aqueous humor outflow ducts 5 extend in fluid communication with and away from the balancing chamber 3, namely the inner volume 4 thereof, and are designed to allow outflow of aqueous humor from the inner volume of the balancing chamber to a subconjunctival space. Thus aqueous humor is transferred from the anterior chamber to the subconjunctival space, and pressure in the anterior chamber is thereby decreased. In the preferred embodiment of the invention, there are three outflow ducts 5, although there may be any number of such ducts greater than one. The inside diameter of the outflow ducts 5 ranges from 20 to 300 micrometers.

In the preferred embodiment of the invention, the balancing chamber 3 comprises an upper wall 3a which delimits the inner volume 4 and faces the underside of the scleral pocket. This upper wall 3a of the balancing chamber 3 comprises a plurality of through holes 3b which establish fluid communication between the inner volume 4 and the outside environment. The number of holes 3b ranges from 4 to 50, preferably from 10 to 40, more preferably from 15 to 25. The diameter of each hole 3b ranges from 2 to 50 micrometers, preferably from 5 to 20 micrometers, and is more preferably about 10 micrometers. Preferably, all the holes 3b have the same diameter. The holes 3b have the purpose of allowing at least partial escape of the aqueous humor contained in the balancing chamber 3, which comes from the anterior chamber of the eyeball and flows toward the scleral portion with the pocket that contains the balancing chamber 3. Such liquid drainage may result from an outflow of liquid through the holes 3b, due to a natural pressure difference between the inner volume 4 and the environment outside it, or may be obtained by creating such pressure difference, e.b. by applying an impulsive force to the balancing chamber 3, to cause a change in the inner volume and a consequent pressure variation. The at least partial escape of aqueous humor through the holes 3b ensures continuous wettability of the scleral portion with the balancing chamber 3, which inhibits or delay scar adhesion between the sclera and the balancing chamber 3.

Advantageously, a retainer member 6 is placed in the feeding duct 2, namely between the first 2a and the second 2b ends thereof, and acts as a backflow-preventing valve for one-way transfer of fluid from the first end 2a to the second end 2b of the feeding duct 2, thereby preventing fluid transfer from the second end 2b to the first end 2a of the feeding duct 2. In other words, the retainer member 5 allows transfer of aqueous humor from the anterior chamber to the exterior of the eye, and prevents any fluid transfer in the opposite direction. Therefore, the retainer member 6 acts as a non-return valve.

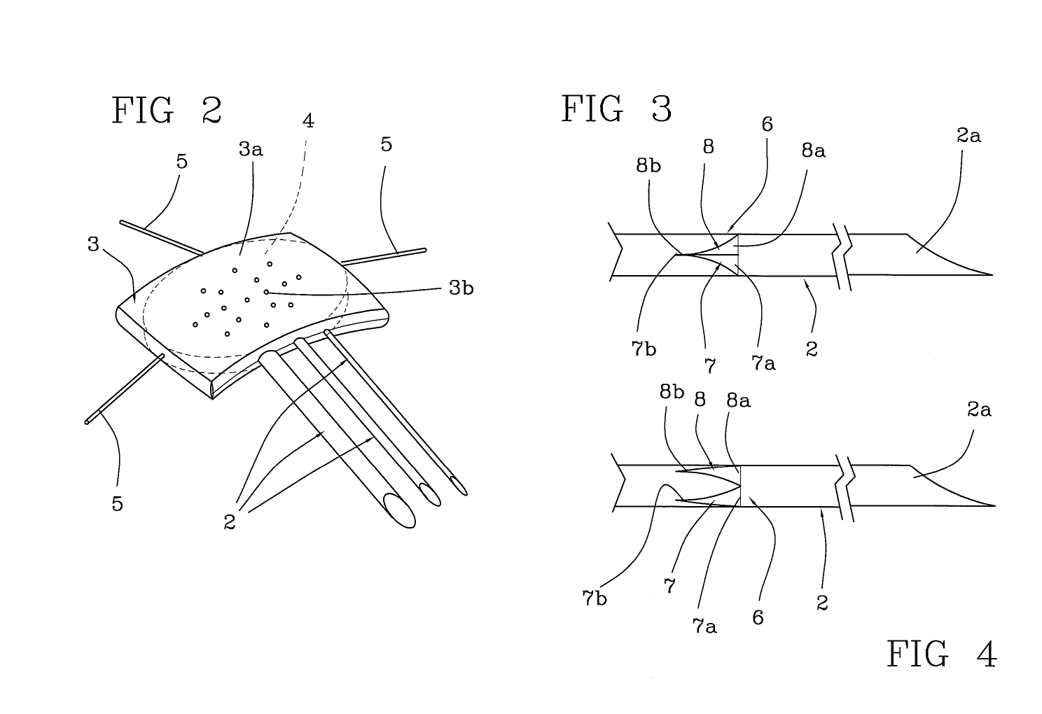

In the preferred embodiment of the invention, the retainer member 6 comprises at least two flaps 7, 8 having respective first edges 7a, 8a attached to inner walls of the feeding duct 2 and respective mutually facing free edges 7b, 8c (see FIGS. 3 and 4) This type of retainer member is functionally and structurally similar to a bicuspid valve, such as the mitral valve. Particularly, the free edges 7b, 8b may be switched from a feeding duct closing state (see FIG. 3), in which the free edges are mutually contacting, and a feeding duct opening state (see FIG. 4), in which the free edges are in spaced relationship. Such switching between the closing and opening state occurs as a result of the pressure difference between the first end 2a and the second end 2b of the feeding duct 2, Particularly, when (aqueous humor) pressure at the first end 2a of the feeding duct is higher than pressure at the second end 2b, the free edges 7b, 8b move apart from each other by elastic deformation, due to the pressure applied to the walls of the two flaps 7, 8. On the other hand, when (aqueous humor) pressure at the second end 2b of the feeding duct is higher than pressure at the first end 2a, the free edges 7b, 8b come close to each other by elastic deformation, thereby closing the feeding duct 2. It shall be noted that the cusp defined by the free ends 7b, 8b of the two flaps 7, 8 faces toward the second end 2b of the feeding duct 2 (as schematically shown in FIG. 3).

Preferably, the pressure difference that can open the two flaps is greater than 100 Pa, whereas the flaps tend to close again with pressure differences of more than 10 Pa.

In an alternative embodiment of the invention, there are three feedin ducts 2, having different inside diameters. Particularly, in this embodiment (see FIG. 2) a first feeding duct has an inside diameter of about 50 micrometers, a second feeding duct has an inside diameter of about 100 micrometers, and a third feeding duct has an inside diameter of about 200 micrometer. Each feeding duct can trigger an outflow of aqueous humor from the anterior chamber to the inner volume of the balancing chamber when the above mentioned pressure difference between the anterior chamber and the inner volume of balancing chamber exceeds predetermined values. For instance, the feeding duct with the greater diameter establishes an outflow at pressure differences of less than the pressure difference that is needed for actuation of the duct with the smaller diameter. Preferably, each feeding duct has a retainer member 6 as described above.

The material of which the device 1 is formed is a biocompatible and hydrophobic acrylic plastic material, having a low wettability, to avoid adhesion of solid particles to the walls of the device. It shall be further noted that the balancing chamber 3 has a substantially curved shape, for better conformation to the natural curvature of the eyeball.

In order to put the device 1 in place, the surgeon will operate as follows. An opening is formed in the superior conjunctiva, preferably close to the limbus, to provide access to the underlying sclera. A trapdoor flap is cut on the sclera to expose an inner portion of the sclera, i.e.

a portion of the sclera is lifted without completely removing such portion. It shall be noted that this flap has a depth of about one third the thickness of the sclera. Preferably the depth (or thickness) of the flap is about 0.2 mm and the dimensions of the flap are compatible with the dimensions of the device 1 (e.g. 5 mm×5 mm).

Then, a button of sclera is cut and removed at the inner portion of the sclera exposed beneath the flap, thereby forming a groove in the sclera. The dimensions of the removed button are compatible with those of the device 1 (e.g. 5 mm×5 mm) and its thickness is about 0.3 mm. Then, the side and rear walls, of the sclera are cut to form appropriate grooves for receiving the outflow ducts.

Next, a calibrated needle is used to form a hole in the limbus at the portion of the sclera that separates the button from the limbus, to reach the anterior chamber. This hole also extends through the trabecular meshwork. A feeding duct 2 of the device 1 is introduced into the hole, such that the first end 2a of the duct 2 penetrates the anterior chamber. Now, the balancing chamber 3 is placed in the groove that was previously formed in the sclera. The outflow ducts are introduced beneath the side and rear scleral grooves, such that the entire device is received within the sclera. Then, the scleral flap is sutured such that the entire valve device is covered thereby. It shall be noted that the device is entirely contained in the sclera and does not contact the conjunctiva. The outflow ducts 5 are placed in the intrascleral space such that the aqueous humor withdrawn from the anterior chamber may flow into this area. Now, the conjunctiva is sutured.

The above disclosure clearly shows that the valve device and the method for surgical treatment of glaucoma can avoid the post-surgical rejection (scarring) problems that were noted in the prior art. Should the conjunctiva exhibit some reactivity, the effectiveness of the valve would not be affected thereby, as the latter does not directly contact the valve device, which is entirely embedded in the sclera. Furthermore, the provision of holes on the upper surface of the balancing chamber ensures constant wettability of the scleral portion with the valve, and further reduces the occurrence of rejection.

Those skilled in the art will obviously appreciate that a number of changes and variants may be made to the above described configurations, to meet incidental and specific needs. All of these variants and changes shall be contemplated in the scope of the invention, as defined in the following claims.

1. A valve device for use in glaucoma surgery, comprising:
at least one aqueous humor feeding duct (2) having a first end (2a) designed to fit into the anterior chamber of an eyeball and into the trabecular meshwork of an eyeball;
a balancing chamber (3), which is in fluid communication with a second end (2b) of said feeding duct (2), said balancing chamber (3) having an inner volume (4) ranging from 2 mm³ to 6 mm³ and being designed to fit into a surgical groove formed in the sclera of an eyeball;
a retainer member (6) placed within said feeding duct (2) and designed to allow fluid transfer from the first end (2a) toward the second end (2b) of the feeding duct (2) and to inhibit fluid transfer from the second end (2b) toward the first end (2a) of the feeding duct (2);
a plurality of aqueous humor outflow ducts (5) which extend in fluid communication with and away from said balancing chamber (3) and are designed to allow outflow of aqueous humor from the balancing chamber (3) to an intrascleral space.

2. A valve device as claimed in claim 1, wherein the balancing chamber (3) comprises a plurality of holes (3b) which establish fluid communication between said inner volume (4) and the outside environment of the balancing chamber (3).

3. A valve device as claimed in claim 2, wherein said through holes (3b) are formed on an upper wall (3a) of the balancing chamber (3).

4. A valve device as claimed in claim 3, wherein the number of holes (3b) ranges from 4 to 50, preferably from 10 to 40, more preferably from 15 to 25.

5. A valve device as claimed in claim 3, wherein the diameter of each hole (3b) ranges from 2 to 50 micrometers, preferably from 5 to 20 micrometers, and is more preferably about 10 micrometers.

6. A valve device as claimed in claim 1, wherein said balancing chamber (3) has peripheral walls defining said inner volume (4) whose thickness ranges from 10 to 50 micrometers.

7. A valve device as claimed in claim 1 , wherein said retainer member (6) comprises at least two flaps (7, 8) having respective first edges (7a, 8a) attached to inner walls of the feeding duct (2) and respective free edges (7b, 8b) in mutually facing relationship; said free edges (7b, 8b) being adapted to be switched between a feeding duct closing state (2) in which the free edges contact each other and a feeding duct opening state (2) in which the free edges are in spaced relationship.

8. A valve device as claimed in claim 7, wherein said free edges (7b, 8b) are in spaced relationship when a pressure difference between the free end (2a) and the second end (2b) of the feeding duct (2) exceeds 100 Pa and contact each other when a pressure difference between the second end (2b) and the first end (2a) of the feeding duct (2) exceeds 10 Pa.

9. A valve device as claimed in claim 1, wherein the inside diameter of the feeding duct (2) ranges from 20 to 100 micrometers, and is preferably about 50 micrometers.

10. A valve device as claimed in claim 1, comprising a plurality of feeding ducts (2).

11. A valve device as claimed in claim 10, comprising three feeding ducts (2), wherein the inside diameters of the ducts are about 50, 100 and 200 micrometers respectively.

12. A valve device as claimed in claim 1, wherein said balancing chamber (3) has a substantially curved shape and is made of a biocompatible and hydrophobic acrylic material.

13. A method for surgical treatment of glaucoma, comprising the steps of:
providing a valve device (1) as claimed in claim 1;
forming a cut in the superior conjunctiva;
forming a trapdoor flap in the sclera to expose an inner portion of the sclera;
removing a button of sclera beneath said trapdoor flap to define a groove in the sclera;
forming a hole in the surgical limbus and introducing the feeding duct (2) of said valve device into said hole, to the anterior chamber;
placing said balancing chamber (3) of the valve device and the outflow ducts (5) within the groove in the sclera;
suturing the trapdoor flap in the sclera to cover the balancing chamber (3);
suturing the conjunctiva.

14. A method as claimed in claim 13, wherein said trapdoor flap in the sclera has dimensions of about 5 mm×5 mm and a thickness of about 0.2 mm and said button of sclera has dimensions of about 5 mm×5 mm and a thickness of about 0.3 mm.
